(19) United States
(12) Patent Application Publication
(10) Pub. No.: US 2004/0214851 A1
(43) Pub. Date: Oct. 28, 2004

(54) Compositions and methods for induction of opioid receptors

(75) Inventors: Woubalem M.R. Birmachu, St. Anthony Village, MN (US); Herbert B. Slade, Woodbury, MN (US); John C. Stolpa, St. Paul, MN (US); Mirjana Urosevic, Zurich (CH)

Correspondence Address: 3M INNOVATIVE PROPERTIES COMPANY, PO BOX 33427, ST. PAUL, MN, 55133-3427, US

(73) Assignee: 3M Innovative Properties Company

(21) Appl. No.: 10/832,737

(22) Filed: Apr. 27, 2004

Related U.S. Application Data
(60) Provisional application No. 60/466,227, filed on Apr. 28, 2003.

(51) Int. Cl. A61K 31/4745

(52) U.S. Cl. 514/292; 514/301; 514/302; 514/303

(57) ABSTRACT

The present invention provides compositions and method for increasing expression of opioid receptors. Generally, the compositions include and opioid receptor inducing compound and, optionally, an opioid receptor ligand. Generally, the methods include contacting a cell with an amount of an opioid receptor inducing compound effective for inducing expression of the opioid receptor and, optionally, contacting the cell with an opioid receptor ligand.

### CROSS-REFERENCE TO RELATED APPLICATION

This application claims priority to U.S. Provisional Application Serial No. 60/466,227, filed Apr. 28, 2003.

### BACKGROUND

Opioid receptors are transmembrane proteins that are expressed in many different types of cells. Opioid receptors can bind endogenous opioids such as, e.g., enkephalin, dynorphins, and β-endorphin, and also can bind opiate alkaloids such as, e.g., morphine, codeine, heroine, and opium. Opioid receptor ligands can influence cell growth, act as neuromodulators or neurotransmitters, affect immune function, provide analgesia and/or sedation, and can affect mental acuity. Therefore, opioid receptors, when activated by binding a ligand, can influence, for example, cell growth, wound healing, pain sensation, and immune function.

Classical opioid receptors (e.g., μ-, κK-, and δ-opioid receptors) may influence pain sensation by binding to opioid ligands (e.g., endogenous opioids or opiate alkaloids) at the site of tissue injury and in certain areas of the spinal cord. Once activated, opioid receptors inhibit the release of inflammatory mediators at the site of tissue injury and from pain-transmitting nerve fibers, thereby suppressing pain receptors. Activated opioid receptors also suppress signal traffic in specialized nerves that carry pain impulses to the spinal cord and brain. One example of a classical opioid receptor is the μ-opioid receptor, which is widely distributed throughout cells of the central nervous system and also is expressed by certain immune cells, e.g., peripheral blood mononuclear cells (PBMCs).

An opioid receptor that is genetically related but functionally distinct from classical opioid receptors is the Opioid Growth Factor Receptor (OGFr). OGFr has, as a natural ligand Opioid Growth Factor (OGF, or [Met⁵]-enkephalin), a negative growth regulator. OGF influences development, cellular renewal, tumor growth, wound healing and angiogenesis: OGF-OGFr interactions have been identified in human embryos, head and neck squamous cell carcinoma, pancreatic adenocarcinoma, colon cancer, renal cancer, neuroblastoma, skin, corneal epithelium, wound healing, and the gastrointestinal tract.

### SUMMARY

It has been found that certain compounds can induce expression of opioid receptors. Accordingly, the present invention provides compositions and methods that include increasing expression of opioid receptors. Such methods may have diagnostic and/or therapeutic utility.

In one aspect, the invention provides a therapeutic combination that includes an ORIC in an amount effective to induce expression of an opioid receptor, and an opioid receptor ligand.

In another aspect, the invention provides a method of increasing a biological activity in response to an opioid receptor ligand. Generally, the method includes contacting a cell with an amount of an opioid receptor inducing compound effective for inducing expression of an opioid receptor, and contacting the cell with an opioid receptor ligand.

Generally, the opioid receptor ligand can be any agonist or antagonist of the opioid receptor. In some embodiments, the opioid receptor ligand can be an opioid peptide such as, for example, Opioid Growth Factor. In other embodiments, the opioid receptor ligand can be an opioid or an opioid alkaloid. In still other embodiments, the opioid receptor ligand can be an antibody capable of binding to the opioid receptor. In certain specific embodiments, an antibody may be conjugated to a cytotoxic moiety.

In another aspect, the present invention provides a method of treating a condition in a subject treatable by inducing expression of an opioid receptor. Generally, the method includes contacting cells capable of expressing an opioid receptor with a therapeutically effective amount of an opioid receptor inducing compound and contacting cells with a therapeutically effective amount of an opioid receptor ligand.

In some embodiments, contacting cells capable of expressing an opioid receptor with an opioid receptor inducing compound can include (a) contacting cells capable of expressing an opioid receptor with the opioid receptor inducing compound in vitro, thereby generating induced cells; and (b) administering at least a portion of the induced cells to the subject. In alternative embodiments, the cells capable of expressing an opioid receptor may be contacted with the opioid receptor inducing compound in vivo.

In yet another aspect, the present invention also provides a method of reducing effects of tissue damage. Generally, the method includes increasing expression of an opioid receptor in cells of damaged tissue by administering an opioid receptor inducing compound to at least a portion of the damaged tissue and contacting an opioid receptor ligand with the damaged tissue.

In some embodiments, the effects of tissue damage reduced by the method include pain, scarring, or both.

Various other features and advantages of the present invention should become readily apparent with reference to the following detailed description, examples, claims and appended drawings. In several places throughout the specification, guidance is provided through lists of examples. In each instance, the recited list serves only as a representative group and should not be interpreted as an exclusive list.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows the induction of Opioid Growth Factor Receptor (OGFr) by an opioid receptor inducing compound (ORIC).

FIG. 2 shows the induction of OGFr by ORIC2, ORIC3, ORIC4, and ORIC5.

FIG. 3 shows the induction of the μ-opioid receptor by ORIC2, ORIC3, ORIC4, and ORIC5.

FIG. 4 shows immunohistochemistry performed on tissue sections originating from sBCC tissue samples, demonstrating expression of OGFr.

FIG. 5 shows expression of OGFr protein in basal cell carcinomas cell line (L1) and human keratinocytes (Ker).

### DETAILED DESCRIPTION OF ILLUSTRATIVE EMBODIMENTS OF THE INVENTION

Certain compounds have been identified as compounds that upregulate expression of opioid receptors—opioid receptor inducing compounds (“ORICs”)—and may therefore influence opioid receptor-mediated biological activity such as, for example, cell growth, immune function, and pain sensation. Such compounds may be used in methods that involve upregulating expression of opioid receptors and, therefore, increasing opioid receptor activity. Increased opioid receptor activity may be manifested by one or more of, for example, increasing the magnitude of an opioid receptor-mediated biological activity, decreasing the threshold amount of opioid receptor ligand required to generate opioid receptor-mediated biological activity, or converting an opioid/opiate alkaloid non-responsive cell to an opioid/opiate alkaloid responsive cell. For example, increasing Opioid Growth Factor Receptor (“OGFr”) activity in certain cell types may decrease or slow the growth rate of those cells. As another example, increasing μ-opioid receptor activity in cells of damaged tissue may reduce pain associated with the tissue damage.

ORICs may be used in compositions capable of, and methods that involve, increasing expression of opioid receptors in order to identify or target a cell. Cells expressing opioid receptors may, for example, be targets for ligands capable of binding the opioid receptor. Thus, increasing expression of an opioid receptor may make cells that express the receptor more readily detectable and/or more susceptible to opioid receptor-targeted therapies or opioid receptor ligand-mediated therapies.

For example, increasing expression of an opioid receptor may be useful for certain immunotherapy or chemotherapy treatments. In some cases, an antibody (i.e., a ligand) specific for an opioid growth factor (i.e., an opioid receptor) may deliver a cytotoxic agent to a cell expressing an increased amount of the opioid receptor. As another example, a fluorchrome-labeled antibody may be used to label or identify a cell expressing an increased amount of the opioid receptor. In yet another example, increased expression of an opioid receptor may make cells expressing the opioid receptor more sensitive and/or responsive to natural ligands such as, for example, OGF.

For purposes of this invention, the following terms shall have the meanings set forth as follows:

“Agonist” refers to a compound that can combine with a receptor to produce a cellular response. For example, agonists of opioid receptors can include endogenous opioid peptides, opiate alkaloids, or both.

“Antagonist” refers to a compound that interferes with the cellular response that can be produced by an agonist.

“Biological activity” refers to any cellular response to agonist-receptor binding (e.g., signal transduction, cell growth, cell maturation, production and/or secretion of proteins or peptides, and the like).

“Express” and variations thereof refer to the conversion of genetic information in a nucleotide sequence to a gene product. Expression of a nucleotide sequence (e.g., a gene) may be measured and/or described with reference to (a) transcription of DNA to mRNA, (b) translation of mRNA to protein, (c) post-translational steps (e.g., modification of the primary amino acid sequence; addition of a carbohydrate, a lipid, a nucleotide, or other moiety to the protein; assembly of subunits; insertion of a membrane-associated protein into a biological membrane; and the like), or any combination of the foregoing.

“Induce” and variations thereof refer to any measurable increase in expression of a gene and/or gene product such as, for example, a protein.

“Ligand” and variations thereof refer to a compound that is capable of binding, to another, specified compound (e.g. a molecule capable of binding to a receptor).

“OGFr-ARF” refers to any putative protein product encoded by an alternate reading frame of an OGFr gene.

“Opioid receptor inducing compound” or “ORIC” refers to a compound that induces expression of at least one opioid receptor (e.g., OGFr) or a putative protein encoded by an alternate reading frame of an opioid receptor gene (e.g., OGFr-ARF).

“Protein” refers to any sequence of two or more amino acid residues without regard to the length of the sequence, as well as any complex of two or more separately translated amino acid sequences. Protein also refers to amino acid sequences chemically modified to include a carbohydrate, a lipid, a nucleotide sequence, or any combination of carbohydrates, lipids, and/or nucleotide sequences. As used herein, “protein,” “peptide,” and “polypeptide” are used interchangeably.

“Prodrug” refers to a derivative of a drug molecule that requires a chemical or enzymatic biotransformation in order to release the active parent drug in the body.

“Treat” or “treatment” or any variation thereof refers to reducing, ameliorating, or resolving, to any extent, the symptoms or signs related to a condition. An ORIC may be used as a primary treatment or as an adjunct to primary treatment, for example, where increased opioid receptor expression potentiates a primary treatment.

Also herein, the recitations of numerical ranges by endpoints include all numbers subsumed within that range (e.g., 1 to 5 includes 1, 1.5, 2, 2.75, 3, 3.80, 4, 5, etc.).

The present invention provides therapeutic combinations that include an ORIC in an amount effective to induce expression of at least one opioid receptor, and an opioid receptor ligand in an amount effective to modulate at least one opioid receptor-mediated biological activity. The ORIC may be any suitable ORIC such as, for example, one of the ORICs described below. The opioid receptor ligand may be any suitable opioid receptor ligand such as, for example, one of the opioid receptor ligands identified below. In certain embodiments, the combination may include two or more ORICs and/or two or more opioid receptor ligands.

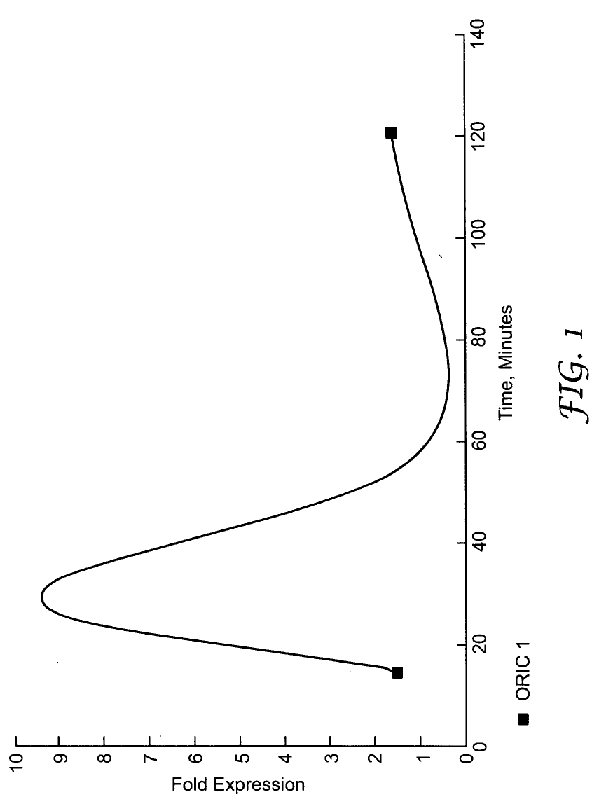

In another aspect, the invention provides a method of increasing the expression of an opioid receptor. Generally, the method includes contacting a cell capable of expressing an opioid receptor with a compound effective for inducing expression of the opioid receptor. Increased expression may be based on (a) mRNA transcription from the opioid receptor gene, (b) opioid receptor protein translated from opioid receptor mRNA, (c) opioid receptor insertion into a membrane of the cell, or any combination of any of the foregoing. For example, FIG. 1 shows the effect of an opioid receptor inducing compound (ORIC) on the level of mRNA transcribed from the opioid receptor gene OGFr, which codes for the Opioid Growth Factor receptor, in human peripheral blood mononuclear cells (PBMCs). OGFr mRNA increased more than nine-fold after PBMCs were incubated in vitro for 15 minutes with an ORIC at a concentration of 5 μM.

The cells used in the method can be any cells capable of expressing an opioid receptor. The cells may be naturally capable of expressing an opioid receptor such as, for example, skin cells, blood cells, neurons, epithelial cells, and the like, as well as tumor cells derived from one of the foregoing. Suitable cells also include cells derived from cells that naturally express an opioid receptor such as, for example, genetically modified cells, hybridomas, and cells of immortalized cell lines. Cells suitable for use in the method also include cells that do not naturally express an opioid receptor, but have been genetically modified so that the cells have acquired the capability of expressing an opioid receptor. The genetic modification may include (a) introduction of a heterologous opioid receptor gene into a cell by transformation, transduction, or transfection; (b) introduction of a homologous opioid receptor gene into a cell by transformation, transduction, or transfection; or (c) expression of a native opioid receptor by removing one or more factors repressing expression of the native opioid receptor gene. Various methods of creating genetically modified cells are known.

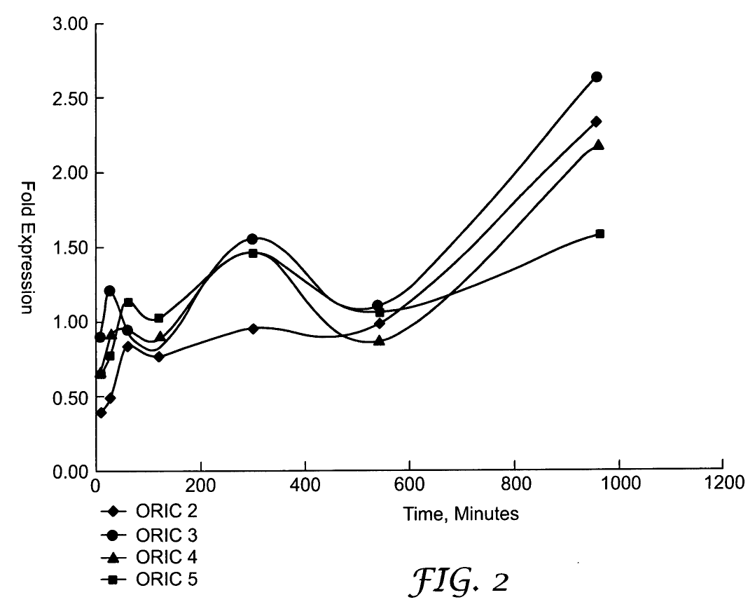

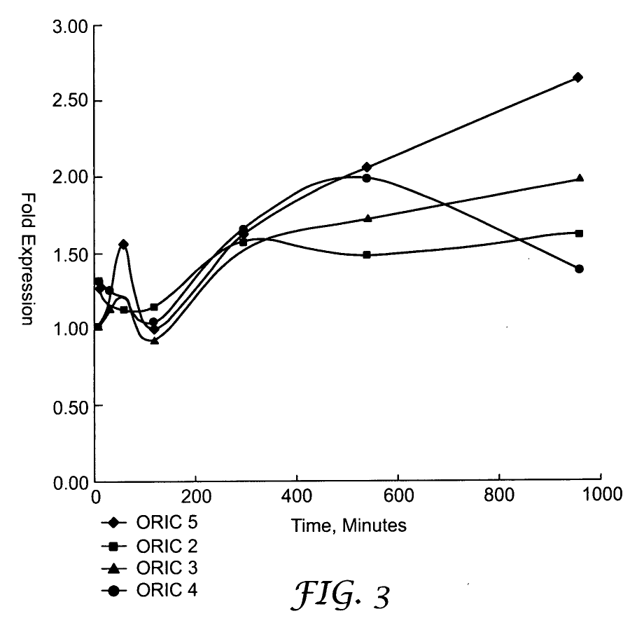

The opioid receptor induced by the method may be any desired opioid receptor, for example, the μ-opioid receptor, the κ-opioid receptor, the δ-opioid receptor, or the ζ-opioid receptor. In certain embodiments, the opioid receptor is the μ-opioid receptor or the ζ-opioid receptor. For example, FIG. 2 shows the effect of four different ORICs on the level of mRNA transcribed from the ζ-opioid receptor gene OGFr in human PBMCs. FIG. 3 shows the effect of four different ORICs on the level of mRNA transcribed from the μ-opioid receptor gene in human PBMCs.

Suitable ORICs are described in detail below. The cells may be contacted with an ORIC either in vitro or in vivo. When the cells are induced in vitro, the cells may be collected from any suitable source including but not limited to a cell culture or a subject. Cells may be contacted with an ORIC in any concentration suitable for inducing the cells to express an opioid receptor. The precise concentration of the ORIC required to induce expression of an opioid receptor in vitro may vary according to factors known in the art including but not limited to the physical and chemical nature of the ORIC, the nature of other components of the culture medium, the length of time the cells are incubated with the ORIC, and the particular cell type being induced. Accordingly, it is not practical to set forth generally the concentration of an ORIC required to induce expression of an opioid receptor for all possible in vitro applications. Those of ordinary skill in the art, however, can readily determine the appropriate amount with due consideration of such factors.

Suitable opioid receptor ligands are described in detail below. The cells may be contacted with an opioid receptor ligand either in vitro or in vivo. When the cells are induced in vitro, the cells may be collected from any suitable source including but not limited to a cell culture or a subject. Cells may be contacted with an opioid receptor ligand in any concentration suitable for binding of the ligand to an opioid receptor. The precise concentration of the opioid receptor ligand required to bind an opioid receptor in vitro may vary according to factors known in the art including but not limited to the physical and chemical nature of the opioid receptor ligand, the nature of other components of the culture medium, the length of time the cells are incubated with the opioid receptor ligand, and the particular cell type being induced. Accordingly, it is not practical to set forth generally the concentration of an opioid receptor ligand required to bind an opioid receptor for all possible in vitro applications. Those of ordinary skill in the art, however, can readily determine the appropriate amount with due consideration of such factors.

In some cases, the method described above can result in cells having one or more biological activities that can provide therapeutic treatment for certain conditions. Accordingly, the present invention also provides a method of treating a condition in a subject that is treatable by inducing expression of an opioid receptor. Generally, the method includes contacting cells capable of expressing an opioid receptor with a therapeutically effective amount of an opioid receptor inducing compound (ORIC) and contacting the cells with a therapeutically effective amount of an opioid receptor ligand.

The cells may be induced in vivo by administering to the subject a therapeutically effective amount of an ORIC. The precise concentration of the ORIC required to induce expression of an opioid receptor in vivo may vary according to factors known in the art including but not limited to the physical and chemical nature of the ORIC, the particular formulation of the ORIC being administered, the route by which the ORIC is being administered, the dosing regimen, the particular cell type being induced, and the desired effect. Accordingly, it is not practical to set forth generally the concentration of an ORIC required to induce expression of an opioid receptor for all possible in vivo applications. Those of ordinary skill in the art, however, can readily determine the appropriate amount with due consideration of such factors.

In some cases, inducing cells to increase expression of an opioid receptor may increase the cells' sensitivity to endogenous levels of an opioid receptor ligand. Thus, in some embodiments, the step of contacting the induced cells with a therapeutically effective amount of an opioid receptor ligand may include merely allowing the induced cells to contact endogenous opioid receptor ligand, without having to provide or otherwise influence the concentration or amount of opioid receptor ligand.

Example 2 describes the administration of an ORIC to subjects for the treatment of superficial basal cell carcinoma (sBCC). Biopsies were taken from each subject before and after treatment with the ORIC. Expression of the OGFr gene increased an average of 2.6-fold after treatment with the ORIC. Suitable therapeutic formulations and dosages for in vivo administration of an ORIC are described in detail below.

In another embodiment, an ORIC may be administered to, for example, reduce or even reverse tumor growth. Opioid Growth Factor receptor (OGFr) is expressed, for example, by sBCC cells. Opioid Growth Factor (OGF), a natural ligand of OGFr, is a negative regulator of cell growth. Inducing expression of OGFr by sBCC cells may make the tumor cells more sensitive to OGF, thereby allowing OGF to more readily or more completely control the growth of the induced cells. Additionally, antibody responses to OGFr have been observed in patients diagnosed with various cancers including, for example, melanoma and chronic myelogenous leukemia. In addition, OGFr expression has been shown in lung cancer, prostate cancer, breast cancer, and ovarian cancer, thus raising the possibility of controlling the growth of such tumors by regulating the expression of OGFr.

Inducing cells in this way may make the cells more sensitive to OGF or an OGF analog such as, for example, (a) endogenous OGF or (b) exogenous OGF or an OGF analog provided as a therapeutic agent. Thus, in some embodiments, the method may further include administering to the subject an OGFr ligand after expression of the OGFr has been increased. Because OGF is a known negative regulator of cell growth, contacting OGF with cells that express OGFr may slow the growth and/or proliferation of the cells (hereinafter, “cell growth”). Increasing the expression of OGFr by the cells before contacting the cells with OGF may slow cell growth to a greater degree that would otherwise occur. Thus, a similar dose of OGF may more potently inhibit cell growth. In addition (or alternatively), a desired amount of cell growth inhibition may be obtained using a smaller dose of OGF, thereby reducing the cost and/or side effects associated with administering OGF.

Alternatively, the cells may be contacted with the ORIC in vitro, thereby creating a population of induced cells. In such embodiments, the induced cells may be introduced into the subject to provide the therapeutic treatment. The cells induced in vitro may be collected from the subject or may be from any suitable source. The induced cells may be administered to the subject in any suitable manner including but not limited to injection (e.g., intravenous, subcutaneous, intraperitoneal, intradermal, etc.), abrasion (e.g., dermal abrasion), or topical suspension.

An opioid receptor ligand may be contacted with a cell expressing an opioid receptor in vivo by administering to the subject a therapeutically effective amount of an opioid receptor ligand. The precise amount of the opioid receptor ligand required to result in the desired biological activity in vivo may vary according to factors known in the art including but not limited to the physical and chemical nature of the opioid receptor ligand, the particular formulation of the opioid receptor ligand being administered, the route by which the opioid receptor ligand is being administered, the dosing regimen, the particular cell type being induced, and the desired effect. Accordingly, it is not practical to set forth generally the concentration of an opioid receptor ligand required bind an opioid receptor for all possible in vivo applications. Those of ordinary skill in the art, however, can readily determine the appropriate amount with due consideration of such factors.

Alternatively, the cells may be contacted with the opioid receptor ligand in vitro. In such embodiments, the cells may be introduced into the subject after being contacted with the opioid receptor ligand to provide the therapeutic treatment. The cells contacted with an opioid receptor ligand in vitro may be collected from the subject or may be from any suitable source. The cells may be administered to the subject in any suitable manner including but not limited to injection (e.g., intravenous, subcutaneous, intraperitoneal, intradermal, etc.), abrasion (e.g., dermal abrasion), or topical suspension.

The present invention also provides a method of reducing the effects of tissue damage. Generally, the method includes increasing the expression of an opioid receptor in cells of damaged tissue by administering an ORIC to at least a portion of the damaged tissue. Effects of tissue damage can include pain and scarring (e.g., the formation and/or development of keloids—benign mesenchymal tumors—at and surrounding a site of injury). Thus, administering an ORIC to damaged tissue can provide pain relief and reduce scarring.

In one embodiment, the ORIC may be administered to damaged tissue to relieve pain associated with the damaged tissue. Additionally, an ORIC may be administered to damaged tissue to reduce or control pain associated with some subsequent treatment. For example, an ORIC may be applied to damaged tissue prior to ablation therapy intended to removed at least a portion of the damaged tissue. Ablation therapies include excision or erosion of tissue, each of which can cause considerable pain. An ORIC may be administered for a period before such therapy to reduce the pain associated with the therapy. In some embodiments, the ORIC may be administered in conjunction with an opioid receptor ligand (e.g., a μ-opioid receptor ligand such as an endogenous opioid or opiate alkaloid) in order to enhance the ability of the opioid receptor ligand to control or reduce pain. The ORIC may be administered before, after, or simultaneous with administration of the opioid receptor ligand.

In an alternative embodiment, an ORIC may be administered to damaged tissue to reduce the formation of keloids associated with scarring and abnormally regulated wound healing. Alternatively, an ORIC may be administered to therapeutically treat an area in which keloids have already formed as a result of abnormally regulated wound healing. Suitable compounds, formulations, and dosages for reducing effects of tissue damage by administering an ORIC to damaged tissue are described in detail below.

In an alternative embodiment, an ORIC may be administered to increase the expression of an opioid receptor and thereby make a tumor cell more sensitive to immunotherapy or chemotherapy treatments. For example, antibodies specific for OGFr may be administered to bind OGFr molecules in tumor cells. Antibody-bound tumor cells may then be more effectively recognized and destroyed by immune cells. As another example, antibodies specific for OGFr may be conjugated to a cytotoxic agent. The conjugated antibodies may be administered to bind tumor cells expressing OGFr and deliver the cytotoxic moiety, thereby destroying the tumor cells.

In some embodiments of the present invention, the compound used to induce opioid receptor expression may be a small molecule compound known to be an immune response modifier (“IRM”). Many small molecule IRM compounds are known to regulate certain immune functions, e.g., cytokine expression. Surprisingly, certain small molecule IRM compounds have now been shown to induce expression of opioid receptors, i.e., act as opioid receptor inducing compounds (“ORICs”).

Small molecule compounds suitable for use as ORICs in the methods of the present invention may have a molecular weight of less than about 1000 Daltons, although in some embodiments the compounds may have a molecular weight of less than about 700 Daltons and in some cases the compounds may have a molecular weight from about 200 Daltons to about 400 Daltons.

In some embodiments of the present invention, a suitable ORIC compound may include a 2-aminopyridine fused to a five membered nitrogen-containing heterocyclic ring, or a 4-aminopyrimidine fused to a five membered nitrogen-containing heterocyclic ring.

In some embodiments of the present invention, a suitable ORIC compound may be an imidazoquinoline amine including but not limited to an amide substituted imidazoquinoline amine, a sulfonamide substituted imidazoquinoline amine, a urea substituted imidazoquinoline amine, an aryl ether substituted imidazoquinoline amine, a heterocyclic ether substituted imidazoquinoline amine, an amido ether substituted imidazoquinoline amine, a sulfonamido ether substituted imidazoquinoline amine, a urea substituted imidazoquinoline ether, a thioether substituted imidazoquinoline amine, or a 6-, 7-, 8-, or 9-aryl or heteroaryl substituted imidazoquinoline amine; a tetrahydroimidazoquinoline amine including but not limited to an amide substituted tetrahydroimidazoquinoline amine, a sulfonamide substituted tetrahydroimidazoquinoline amine, a urea substituted tetrahydroimidazoquinoline amine, an aryl ether substituted tetrahydroimidazoquinoline amine, a heterocyclic ether substituted tetrahydroimidazoquinoline amine, an amido ether substituted tetrahydroimidazoquinoline amine, a sulfonamido ether substituted tetrahydroimidazoquinoline amine, a urea substituted tetrahydroimidazoquinoline ether, or a thioether substituted tetrahydroimidazoquinoline amine; an imidazopyridine amine including but not limited to an amide substituted imidazopyridine amine, a sulfonamido substituted imidazopyridine amine, a urea substituted imidazopyridine amine, an aryl ether substituted imidazopyridine amine, a heterocyclic ether substituted imidazopyridine amine, an amido ether substituted imidazopyridine amine, a sulfonamido ether substituted imidazopyridine amine, a urea substituted imidazopyridine ether, or a thioether substituted imidazopyridine amine; a 1,2-bridged imidazoquinoline amine; a 6,7-fused cycloalkylimidazopyridine amine; an imidazonaphthyridine amine; a tetrahydroimidazonaphthyridine amine; an oxazoloquinoline amine; a thiazoloquinoline amine; an oxazolopyridine amine; a thiazolopyridine amine; an oxazolonaphthyridine amine; a thiazolonaphthyridine amine; or a 1H-imidazo dimer fused to a pyridine amine, a quinoline amine, a tetrahydroquinoline amine, a naphthyridine amine, or a tetrahydronaphthyridine amine.

In certain embodiments, a suitable compound may be 1-(2-methylpropyl)-1H-imidazo[4,5-c]quinolin-4-amine. In other embodiments, a suitable compound can include a 6,7-fused cycloalkylimidazopyridine amine such as, for example, 4-amino-2-(ethoxymethyl)-α,α-dimethyl-6,7,8,9-tetrahydro-1H-imidazo[4,5-c]quinoline-1-ethanol. In other embodiments, a suitable compound can include a thiazoloquinoline amine such as, for example, 2-propylthiazolo[4,5-c]quinolin-4-amine. In other embodiments, a suitable compound can include a sulfonamide substituted imidazoquinoline amine such as, for example, N-[4-(4-amino-2-butyl-1H-imidazo[4,5-c]quinolin-1-yl)butyl]methanesulfonamide. In still other embodiments, a suitable compound can include a urea substituted tetrahydroimidazoquinoline amine such as, for example, N-[4-(4-amino-2-methyl-6,7,8,9,-tetrahydro-1H-imidazo[4,5-c]quinolin-1-yl)butyl]morpholine-4-carboxamide.

In certain embodiments, the IRM compound may be an imidazonaphthyridine amine, a tetrahydroimidazonaphthyridine amine, an oxazoloquinoline amine, a thiazoloquinoline amine, an oxazolopyridine amine, a thiazolopyridine amine, an oxazolonaphthyridine amine, or a thiazolonaphthyridine amine.

In certain embodiments, the IRM compound may be a substituted imidazoquinoline amine, a tetrahydroimidazoquinoline amine, an imidazopyridine amine, a 1,2-bridged imidazoquinoline amine, a 6,7-fused cycloalkylimidazopyridine amine, an imidazonaphthyridine amine, a tetrahydroimidazonaphthyridine amine, an oxazoloquinoline amine, a thiazoloquinoline amine, an oxazolopyridine amine, a thiazolopyridine amine, an oxazolonaphthyridine amine, or a thiazolonaphthyridine amine.

As used herein, a substituted imidazoquinoline amine refers to an amide substituted imidazoquinoline amine, a sulfonamide substituted imidazoquinoline amine, a urea substituted imidazoquinoline amine, an aryl ether substituted imidazoquinoline amine, a heterocyclic ether substituted imidazoquinoline amine, an amido ether substituted imidazoquinoline amine, a sulfonamido ether substituted imidazoquinoline amine, a urea substituted imidazoquinoline ether, a thioether substituted imidazoquinoline amines, or a 6-, 7-, 8-, or 9-aryl or heteroaryl substituted imidazoquinoline amine. As used herein, substituted imidazoquinoline amines specifically and expressly exclude 1-(2-methylpropyl)-1H-imidazo[4,5-c]quinolin-4-amine and 4-amino-α,α-dimethyl-2-ethoxymethyl-1H-imidazo[4,5-c]quinolin-1-ethanol.

Unless otherwise indicated, reference to a compound can include the compound in any pharmaceutically acceptable form, including any isomer (e.g., diastereomer or enantiomer), salt, solvate, polymorph, and the like. In particular, if a compound is optically active, reference to the compound can include each of the compound's enantiomers as well as racemic mixtures of the enantiomers.

Additional examples of small molecule compounds that may be suitable for use as ORICs in the methods of the present invention include purine derivatives (such as those described in U.S. Pat. Nos. 6,376,501, and 6,028,076), imidazoquinoline amide derivatives (such as those described in U.S. Pat. No. 6,069,149), 1H-imidazopyridine derivatives (such as those described in Japanese Patent Application 9-255926, U.S. Pat. No. 6,518,265, and European Patent Application EP 1 256 582) and benzimidazole derivatives (such as those described in U.S. Pat. No. 6,387,938).

Examples of small molecule ORIC compounds that include a 4-aminopyrimidine fused to a five membered nitrogen-containing heterocyclic ring include adenine derivatives (such as those described in U. S. Pat. Nos. 6,376,501; 6,028,076; and 6,329,381; and in WO 02/08595).

Other compounds that may be suitable for use as ORICs in a method of the invention include large biological molecules such as oligonucleotide sequences. Some suitable oligonucleotide sequences contain cytosine-guanine dinucleotides (CpG) and are described, for example, in U.S. Pat. Nos. 6,1994,388; 6,207,646; 6,239,116; 6,339,068; and 6,406,705. Some CpG-containing oligonucleotides can include synthetic immunomodulatory structural motifs such as those described, for example, in U.S. Pat. Nos. 6,426,334 and 6,476,000. Other suitable nucleotide sequences lack CpG and are described, for example, in International Patent Publication No. WO 00/75304. Still other suitable compounds include biological molecules such as aminoalkyl glucosaminide phosphates (AGPs) and are described, for example, in U.S. Pat. Nos. 6,113,918; 6,303,347; 6,525,028; and 6,649,172.

Suitable compounds for use as an opioid receptor ligand include any compound capable of binding to an opioid receptor. In some embodiments, the ligand may be a protein (including, e.g., a peptide), a carbohydrate, a nucleic acid, or a lipid. In other embodiments, the ligand may be a small, synthetic chemical compound capable of binding to an opioid receptor.

In some embodiments of the present invention, the compound used as an opioid receptor ligand may be a natural biological ligand for an opioid receptor such as, for example, an opioid peptide. In some embodiments, the opioid receptor ligand may be Opioid Growth Factor. In alternative embodiments, the opioid receptor ligand may be an opioid such as, for example, enkaphalin, a dynorphin, or β-endorphin. In still other embodiments, the opioid receptor ligand may be an opioid alkaloid such as, for example, morphine, heroine, codeine, or opium.

In other embodiments, the compound used as an opioid receptor ligand may be a protein capable of binding to an opioid receptor. In some embodiments, for example, the ligand may be an antibody capable of binding to an opioid receptor. The binding of the ligand to the opioid receptor may or may not induce a biological response mediated by the opioid receptor.

In some embodiments, the opioid receptor ligand may contain an additional chemical moiety that provides, for example, detection, biological, or chemical function. In one embodiment, for example, the ligand may contain a cytotoxic moiety. In other embodiments, the ligand may contain an immunomodulatory compound, such as, for example, a complement molecule. In additional embodiments, the ligand may contain a compound capable of augmenting the immune response.

In some embodiments, the ORIC and one or more opioid receptor ligands may be considered a combination such as, for example, a therapeutic combination. Components of such a combination may be said to be delivered “in combination” with one another if the components are provided in any manner that permits the biological effect of contacting one component with cells to be sustained at least until another. component is contacted with the cells. Thus, components may be delivered in combination with one another even if they are provided in separate formulations, delivered via different routes of administration, and/or administered at different times. For example, an ORIC and an opioid receptor ligand may be considered to be administered “in combination” with one another if the ORIC is administered in a first formulation and the opioid receptor ligand is administered in a second formulation and at a different time that the ORIC, but administered so that the opioid receptor ligand is able to contact cells that express the opioid receptor at an increased level promoted by administration of the ORIC.

The ORIC and or opioid receptor ligand may be provided in any formulation suitable for administration to a subject. Formulations suitable for delivery of ORICs are described, for example, in U.S. Pat. No. 5,736,553; U.S. Pat. No. 5,238,944; U.S. Pat. No. 5,939,090; U.S. Pat. No. 6,365,166; U.S. Pat. No. 6,245,776; U.S. Pat. No. 6,486,186; European Patent No. EP 0 394 026; and U.S. patent Publication No. 2003/0199538.

The compound (whether an ORIC or opioid receptor ligand) may be provided in any suitable form including but not limited to a solution, a suspension, an emulsion, or any form of mixture. The compound may be delivered in formulation with any pharmaceutically acceptable excipient, carrier, or vehicle. For example, the formulation may be delivered in a conventional topical dosage form such as, for example, a cream, an ointment, an aerosol formulation, a non-aerosol spray, a gel, a lotion, and the like. The formulation may further include one or more additives including but not limited to adjuvants, skin penetration enhancers, colorants, fragrances, flavorings, moisturizers, thickeners, and the like.

Moreover, a formulation may be designed to provide certain desirable delivery characteristics such as, for example, depot, targeted accumulation, and/or extended release. Formulations providing such characteristics are described, for example, in U.S. patent application Ser. No. 10/821,330, filed Apr. 9, 2004; Ser. No. 10/821,335, filed Apr. 9, 2004; Ser. No. 60/544,561, filed Feb. 13, 2004; and Ser. No. 60/560,862, filed Apr. 9, 2004.

A formulation containing one or more components of an ORIC-opioid receptor ligand combination may be administered in any suitable manner such as, for example, non-parenterally or parenterally. As used herein, non-parenterally refers to administration through the digestive tract, including by oral ingestion. Parenterally refers to administration other than through the digestive tract such as, for example, intravenously, intramuscularly, transdermally, subcutaneously, transmucosally (e.g., by inhalation), or topically.

The composition of a formulation suitable for practicing the invention will vary according to factors known in the art including but not limited to the physical and chemical nature of the ORIC, the nature of the carrier, the intended dosing regimen, the method of administering the ORIC, whether the ORIC is being delivered in combination with an opioid receptor ligand, and the species to which the formulation is being administered. Accordingly, it is not practical to set forth generally the composition of a formulation effective for all possible applications. Those of ordinary skill in the art, however, can readily determine an appropriate formulation with due consideration of such factors.

In some embodiments, the methods of the present invention include administering an ORIC to a subject in a formulation of, for example, from about 0.001% to about 10% ORIC (unless otherwise indicated, all percentages provided herein are weight/weight with respect to the total formulation) to the subject, although in some embodiments the ORIC may be administered using a formulation that provides ORIC compound in a concentration outside of this range. In certain embodiments, the method includes administering to a subject a formulation that includes from about 0.01% to about 5% ORIC, for example, a formulation that includes about 5% ORIC.

The amount of an opioid receptor agonist, when present, in a formulation suitable for practicing the invention will vary according to factors known in the art including but not limited to the physical and chemical nature of the opioid receptor agonist, the physical and chemical nature of the ORIC included in the combination, the nature of the carrier, the intended dosing regimen, the method of administering the opioid receptor ligand, and the species to which the formulation is being administered. Accordingly, it is not practical to set forth generally the amount of opioid receptor ligand effective for all possible applications. Those of ordinary skill in the art, however, can readily determine an appropriate formulation with due consideration of such factors.

When cells are contacted with the combination of an ORIC and an opioid receptor ligand, each component of the combination may be provided in a single formulation that includes all of the components. Alternatively, the combination may be provided in two or more formulations, each of which may contain one or more components of the combination or together with one or both of the other components. If the combination is provided in a plurality of formulations, the various formulations may be of similar or dissimilar composition. Furthermore, each formulation may be of similar or dissimilar form (e.g., aerosol, gel, cream, solution, etc.) and may be administered via similar or dissimilar delivery routes (e.g., injection, transdermal, intravenous, etc). Also, if the components of the combination are provided in a plurality of formulations, the various components may be contacted with the cells in any order.

An amount of ORIC effective for inducing opioid receptor expression is an amount sufficient to increase one or more of: (a) transcription of mRNA from a structural gene encoding an opioid receptor, (b) translation of the mRNA, and (c) the amount of an opioid receptor inserted into a biological membrane. The precise amount of ORIC required to induce opioid receptor expression will vary according to factors known in the art including but not limited to the physical and chemical nature of the ORIC, the nature of the carrier, the intended dosing regimen, the baseline opioid receptor expression level of the cells to which the ORIC is being administered, the method of administering the ORIC, and the species to which the ORIC is being administered. Accordingly, it is not practical to set forth generally the amount that constitutes an amount of ORIC effective for inducing opioid receptor expression for all possible applications. Those of ordinary skill in the art, however, can readily determine the appropriate amount with due consideration of such factors.

In some embodiments, the methods of the present invention include administering sufficient ORIC to provide a dose of, for example, from about 100 ng/kg to about 50 mg/kg to the subject, although in some embodiments the methods may be performed by administering ORIC in amounts outside this range. In some of these embodiments, the method includes administering sufficient ORIC to provide a dose of from about 10 μg/kg to about 5 mg/kg to the subject, for example, from about 100 μg/kg to about 1 mg/kg.

An effective amount of opioid receptor ligand is an amount sufficient, when contacted with cells that express an opioid receptor, to result in a measurable biological activity or an amount sufficient to bind the opioid receptor and be detected by assay. Contacting the opioid receptor ligand with the opioid receptor may be detected by, for example, a measurable increase or decrease in cell death, a measurable increase or decrease in cell apoptosis, a measurable increase or decrease in cell growth, a measurable increase or decrease in cell proliferation, a measurable increase or decrease in angiogenesis, a measurable decrease in pain, a measurable increase in wound healing, or a measurable decrease in inflammation. Contacting the opioid receptor ligand to the opioid receptor also may be determined by a conventional assay, such as, for example, flow cytometry. The precise amount of opioid receptor ligand required to contact an opioid receptor and result in a measurable biological activity will vary according to factors known in the art including but not limited to the physical and chemical nature of the opioid receptor ligand, the nature of the carrier, the intended dosing regimen, the baseline opioid receptor expression level of the cells to which the opioid receptor ligand is being administered, the method of administering the opioid receptor ligand, and the species to which the opioid receptor ligand is being administered. Accordingly, it is not practical to set forth generally the amount that constitutes an amount of opioid receptor ligand effective for binding an opioid receptor for all possible applications. Those of ordinary skill in the art, however, can readily determine the appropriate amount with due consideration of such factors.

The dosing regimen may depend at least in part on many factors known in the art including but not limited to the physical and chemical nature of the ORIC or the opioid receptor ligand, the nature of the carrier, the amount of ORIC or opioid receptor ligand being administered, the baseline opioid receptor expression level of the cells to which the ORIC is being administered, the method of administering the ORIC or the opioid receptor ligand, and the species to which the ORIC or the opioid receptor ligand is being administered. Accordingly it is not practical to set forth generally the dosing regimen effective to induce opioid receptor expression for all possible applications. Those of ordinary skill in the art, however, can readily determine the appropriate amount with due consideration of such factors.

In some embodiments of the invention, the ORIC or the opioid receptor ligand may be administered on an “as needed” basis. Alternatively, the ORIC or the opioid receptor ligand may be administered on a regular schedule, for example, from about once per week to about twice per day, although in some embodiments the methods of the present invention may be performed by administering the ORIC or the opioid receptor ligand at a frequency outside this range. In certain embodiments, the ORIC or the opioid receptor ligand may be administered from about once every other day to about once per day. In one particular embodiment, the ORIC or the opioid receptor ligand is administered once per day, five days per week.

The methods of the present invention may be performed on any suitable subject. Suitable subjects include but are not limited to animals such as but not limited to humans, non-human primates, rodents, dogs, cats, horses, pigs, sheep, goats, or cows.

### EXAMPLES

The following examples have been selected merely to further illustrate features, advantages, and other details of the invention. It is to be expressly understood, however, that while the examples serve this purpose, the particular materials and amounts used as well as other conditions and details are not to be construed in a matter that would unduly limit the scope of this invention.

Opioid Receptor Inducing Compounds

The ORICs used in the examples are shown in Table 1.

TABLE 1

| Compound | Chemical Name | Reference |
|---|---|---|
| ORIC 1 | 1-(2-methylpropyl)-1H-imidazo[4,5-c] | U.S. Pat. No. |
| | quinolin-4-amine | 4,689,338 |
| | | Example 99 |
| ORIC 2 | 4-amino-2-(ethoxymethyl)-α,α-dimethyl- | U.S. Pat. No. |
| | 6,7,8,9-tetrahydro-1H-imidazo[4,5-c] | 5,352,784 |
| | quinoline-1-ethanol | Example 91 |
| ORIC 3 | 2-propylthiazolo[4,5-c]quinolin-4-amine | U.S. Pat. No. |
| | | 6,110,929 |
| | | Example 12 |
| ORIC 4 | N-[4-(4-amino-2-butyl-1H-imidazo[4,5-c] | U.S. Pat. No. |
| | quinolin-1-yl)butyl]methanesulfonamide | 6,331,539 |
| | | Example 6 |
| ORIC 5 | N-[4-(4-amino-2-methyl-6,7,8,9,- | U.S. Pat. No. |
| | tetrahydro-1H-imidazo[4,5-c]quinolin-1- | 6,573,273 |
| | yl)butyl]morpholine-4-carboxamide | Example 170 |

### Example 1

Human peripheral blood mononuclear cells (PBMCs) were isolated from 240 mL human blood by gradient centrifugation using Histopaque-1077 (Sigma Chemical Company, St. Louis, Mo.). 3.8% sodium citrate was added to the fresh blood at a 1:10 dilution to prevent clotting.

Opioid receptor inducing compounds (ORICs) were dissolved in dimethyl sulfoxide (DMSO, Sigma Chemical Company) at 5 mM concentration. PBMCs were incubated for one hour in X-VIVO 20 medium (Cambrex Bio Science, Walkersville, Md.) at 37° C. in a 5% CO₂ incubator prior to incubation with ORIC prepared in DMSO or with DMSO alone (as a control). The amount of DMSO was kept below 0.1% of the total incubation volume. The final concentrations of ORICs used were 5 μM ORIC 1, 1 μM ORIC 2, 5 μM ORIC 3, 0.1 μM ORIC 4, and 1 M ORIC 5.

PBMCs were incubated for 15, 30, 60, 120 minutes with ORIC 1. PBMCs were incubated with ORIC 2, ORIC 3, ORIC 4, and ORIC 5 for 10 minutes, 30 minutes, 1 hour, 2 hours, 5 hours, 9 hours, or 16 hours. DMSO-treated control samples were incubated for 1 hour, 2 hours, 5 hours, 9 hours, and 16 hours.

At the end of the specified incubation time, cells were lysed and total RNA extracted using the Qiagen RNeasy MidiKit (Qiagen, Inc., Valencia, Calif.). RNA was quantified using absorbance at 260. Quality of RNA was determined from the ratio of absorbance measured at OD260 and OD280 and from the ratio of 18S and 28S bands in RNA 6000 Nano Chip gels run on the Agilent 2100 Bioanalyzer (Agilent Technologies, Inc., Palo Alto, Calif.). All samples had ratios of 1.9 to 2.1 for OD260/280 and a ratio near 2 for the ratio of 28S to 18S in the gels.

RNA was reverse transcribed to cDNA using a Superscript cDNA synthesis kit (Invitrogen Corporation, Carlsbad, Calif.) using an HPLC-purified T7-(dT)₂₄ primer (Genset SA, Paris, France). Labeled cRNA was prepared from double-stranded cDNA by in vitro transcription using an Enzo BioArray High Yield RNA Transcript Labeling Kit in the presence of biotin-11-CTP and biotin-16-UTP (Enzo Diagnostics, Inc., Farmingdale, N.Y.), and purified using an RNeasy MiniKit (Qiagen, Inc.). 10 or 15 μg of biotinylated cRNA were fragmented and hybridized to HG-U95A or U133A GeneChip® arrays (Affymetrix, Santa Clara, Calif.) containing probe sets representing 12,000 genes (HG-U95A) or 22,000 genes (U133A). Chips were hybridized, washed, and stained according to the Affymetrix protocols.

Images were scanned using the Agilent Gene Array Scanner (Agilent Technologies, Inc., Palo Alto, Calif.), and processed using Affymetrix Microarray Suite (MS 4.0 or MS 5.0) software. The total fluorescence intensity of each image was scaled to 500 to enable comparison of different images. The image at each time point was analyzed relative to the appropriate control in order to calculate changes in expression for each gene. Changes in gene expression were deemed significant if the p value for detection and change in expression were <0.05.

The induction of OGFr by ORIC1 is shown in FIG. 1. The induction of OGFr by ORIC2, ORIC3, ORIC4, and ORIC5 are shown in FIG. 2. The induction of the μ-opioid receptor by ORIC2, ORIC3, ORIC4, and ORIC5 are shown in FIG. 3.

### Example 2

Subjects provided a biopsy specimen that was examined for confirmation of superficial basal cell carcinoma (sBCC). ORIC1 was provided in a 5% cream in a formulation shown in Table 1, on a percentage weight-by-weight basis.

TABLE 1

| | Formulation |
|---|---|
| Components | (% w/w) |
| ORIC1 | 5.0 |
| Isostearic Acid | 25.0 |
| Benzyl Alcohol | 2.0 |
| Cetyl Alcohol | 2.2 |
| Stearyl Alcohol | 3.1 |
| White Petrolatum | 3.0 |
| Polysorbate 60 | 3.4 |
| Sorbitan Monostearate | 0.6 |
| Glycerin | 2.0 |
| Methyl Paraben | 0.2 |
| Propyl Paraben | 0.02 |
| Water | 52.98 |
| Xanthan Gum | 0.5 |

The formulation was prepared according to the methods described in U.S. Pat. No. 5,238,944. The final formulation had a pH of5.1, and a viscosity (cps) of0.33×10⁵.

Subjects topically applied the cream to sBCC once daily five times per week for a maximum of six weeks. When the tumor began to show signs of erosion, the tumor was surgically excised.

Total RNA was isolated using the TRIzol reagent (Invitrogen AG, Basel, Switzerland). Double-stranded cDNA was generated using a Superscript cDNA synthesis kit (Invitrogen AG) using an oligo(dT)₂₄ primer containing a T7 RNA polymerase promoter at the 3′ end (Microsynth GmbH, Balgach, Switzerland). Labeled cRNA was prepared from double-stranded cDNA by in vitro transcription using a T7 RNA polymerase (MEGAscript T7 kit, Ambion (Europe) Ltd., Huntingdon, UK) in the presence of biotin-11-CTP and biotin-16-UTP (Enzo Diagnositcs, Inc., Farmingdale, N.Y.), and purified using an RNeasy column (Qiagen AG, Basel, Switzerland). 15 /μg of biotinylated cRNA was fragmented and hybridized to HG-U95A GeneChip® arrays (Affymetrix, Santa Clara, Calif.), which contained a probe set representing about 12,000 genes. Chip hybridization, washing, and staining were performed according to Affymetrix protocols.

Images were scanned and then processed using Affymetrix Microarray Suite 5.0 software. The total fluorescence intensity of each image was scaled to 500 to enable comparison of different images.

### Example 3

Paraffin-embedded tissue sections originating from sBCC tissue samples used for microarray analysis were stained with anti-human OGFR (Mollick et al., Cancer Immunity, 3:3, (2003)). Immunohistochemistry was performed with 1:30 working antibody dilution using alkaline phosphatase-anti-alkaline-phosphatase technique Briefly, 3- to 5-μm-thick tissue sections adherent to slides coated with 0. 1% (wt/v) poly-L-lysine were deparaffinized with xylene and rehydrated. Tissue sections were incubated with an excess of the anti-OGFr antibody for 60 minutes. This was followed by 3 cycles of sequential incubations with rabbit antimouse IgG xenoantibodies and alkaline phosphatase-anti-alkaline phosphatase complexes (DAKO, Glostrup, Denmark). Incubations in the first cycle were of 30-minute duration; the second and third cycles were 10 minutes each. All incubations were performed at room temperature in a moist chamber. The immunoreaction was visualized with a developing solution containing neufuchsin (DAKO). Finally, sections were counterstained with 1% hematoxylin.

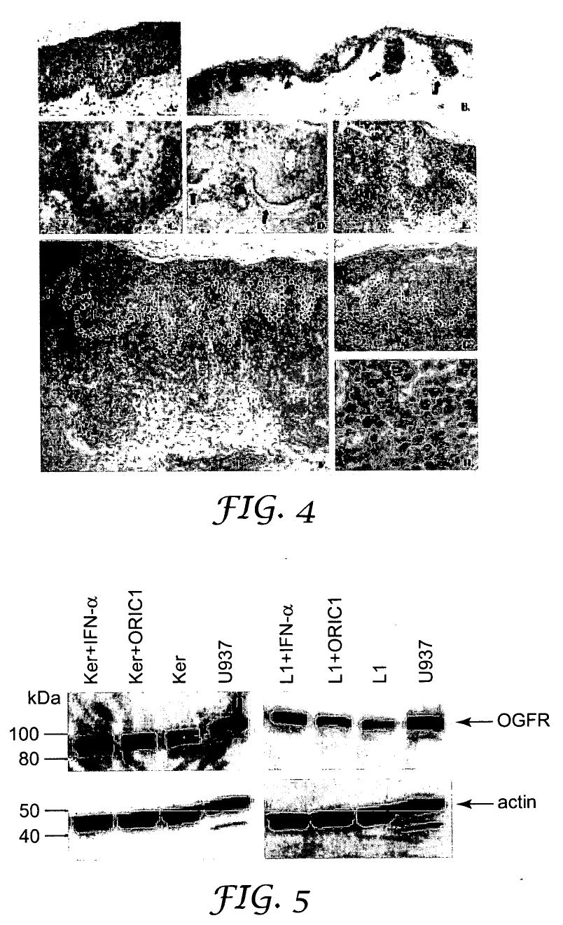

Results are shown in FIG. 4. Panel A. The pattern of OGFr expression in normal epidermis showing clear nuclear OGFr immunoreactivity in the basal, spinous, and, in part, granular cell layer (original magnification (o.m.)×20). Panel B. Cytoplasmic OGFr protein expression in sBCC before IRM treatment. Tumor islets are indicated by arrows. Note the nuclear pattern of OGFr expression in neighboring epidermis, which is not present in tumor islets (o.m.×10). Panel C. OGFr expression in Bowen's disease (o.m×40). Panel D. The absence of OGFr expression in sBCC before ORIC treatment (o.m.×10). Panels E, F, and G. Upregulation of OGFr immunoreactivity in sBCC after ORIC treatment (o.m.×10). For the better discrimination of the infiltrate, tumor borders are accentuated by dotted black line. Panel H. Nuclear OGFr expression in BCC tumor cells after ORIC treatment (o.m.×100).

### Example 4

Following the RNA extraction from the cell lines using TRIzol reagent, proteins were isolated from the intermediate fraction according to the manufacturer's instructions. For immunoblotting analysis, protein extracts were electrophoresed using NOVEX Tris-Glycine gels (Invitrogen AG, Basel, Switzerland) and the buffer system of Lemmli, and subsequently transferred to nitrocellulose an a 12.5 mM Tris base, 100 mM glycine in 20% methanol transfer buffer.

The membranes were blocked with 10 mM Tris, 150 mM NaCl, 5% nonfat powdered milk, 0.25% Tween 20 and incubated for 60 minutes with primary antibody, either anti-OGFr (Mollick et al., Cancer Immunity, 3:3, (2003), 0.25 μg/mL) or anti-actin (clone I-19-R, rabbit polyclonal IgG, Santa Cruz Biotechnology, Inc., Heidelberg, Germany). After incubation with the primary antibody, the membrane was washed extensively with 10 mM Tris-HCl, 150 mM NaCl, 0.25% Tween 20, and then incubated for 45 minutes with the secondary antibody (Goat anti-rabbit IgG(H+L)-HRP conjugate, BioRad Laboratories, Munich, Germany) The blots were developed with chemiluminescence (ECL, Amersham Biosciences, Freiberg, Germany).

Results are shown in FIG. 5. Expression of OGFr protein in basal cell carcinomas cell line (L1) and human keratinocytes (Ker). Cell lines have been treated either with interferon-α (IFN-α) or ORIC1 for 24 hours and then used for protein extraction. U937 cell line represents a positive control for OGFr expression.

### Example 5

C57/BL6 mice are injected on Day 0 intravenously with melanoma cell line B16-F10 (5×10⁵). On day 5 and lasting through day 19, mice are injected intraperitoneally every other day with either (a) PBS (control), (b) 10 mg/kg of ORIC2, (c) 10 mg/kg [Met⁵]-enkephalin, or (d) both.

On Day 20, half the mice are sacrificed and the lungs are removed and weighed. Percent inhibition of lung tumor growth is determined according to the formula:

Placebo weight−Treatment weight×100 Placebo weight

Treatment with either ORIC2 or the combination of ORIC2 and [Met⁵]-enkephalin results in increased inhibition of tumor growth in the lung as compared to the placebo-treated mice.

The other half of the mice are monitored for survival through Day 75. Treatment with either ORIC2 or the combination of ORIC2 and [Met⁵]-enkephalin results in enhanced survival as compared to the placebo-treated mice.

### Example 6

Human subjects with lung cancer are injected intravenously with a placebo (control), ORIC5 (10 mg/kg), or both ORIC5 (10 mg/kg) and OGF (1 mg/kg) three times a week for eight weeks. Tumor size is measured by CT scan. At eight weeks, the tumor size is measurably smaller after treatment with ORIC5 than prior to treatment. Tumor size after treatment with the combination of ORIC5 and OGF is measurably smaller than prior to treatment.

Additionally, the five-year survival rate of subjects treated with ORIC5 is greater than the five-year survival rate of the control group. The five-year survival rate of subjects treated with ORIC5 and OGF also is greater than the five-year survival rate of the control group.

### Example 7

Cells from the colon adenocarcinoma cell line HT-29 (TACC, Manassas, Va.) are incubated at 37° C. in a 96 well plate (5×10⁵ cells/ well) in McCoy's 5a medium (1.5 mM L-glutamine, 90%; fetal bovine serum, 10%) on Day 0. On Day 1, cells are stimulated with media (control), ORIC3 (1 μg/mL), [Met5]-enkephalin (10 μg/mL), or both ORIC3 (1 μg/mL) and [Met5]-enkephalin (10 μg/mL) overnight. On Day 2, apoptotic cells are detected using a terminal deoxynucleotidyl transferase-mediated dUTP nick end labeling (TUNEL) assay. Treatment with either ORIC3 or the combination of ORIC3 and [Met⁵]-enkephalin results in greater pro-apoptotic effects than the control.

### Example 8

A topical formulation of ORIC1 is applied to melanoma lesions of patients three times per week for two weeks. Tumor cells are excised from patients, disaggregated and suspended in PBS/BSA (approximately 10⁶ per mL). The cells are fixed by adding an equal volume of PBS containing 4% formaldehyde and incubating for 20 minutes in the dark at room temperature. Following two washes in PBS/BSA, the cells are permeabilized by incubation in PBS/1% Triton for 10 minutes in the dark at room temperature.

Antibodies specific for OGFr-ARF (Mollick et al., Cancer Immunity 3:3 (2003)) are added and the cells are incubated for 30 minutes in the dark at room temperature. Following two PBS/BSA washes the cells are incubated in PBS/BSA containing FITC-conjugated anti-human IgG (Amersham Biosciences, Piscataway, N.J.) for 30 minutes in the dark. The cells are then washed twice in PBS/BSA and resuspended in PBS/BSA. The amount of OGFr-ARF expressed in the cells is then determined by analysis on a flow cytometer.

### Example 9

OGFr-ARF peptides (Mollick et al., Cancer Immunity 3:3 (2003)) are adhered to ELISA plate wells (approximately 0.5 μg peptide/well) overnight in a carbonate buffer (pH 9.5). The wells are washed with PBS and blocked for 2 hours with PBS/BSA. Serum from patients is diluted in PBS/BSA, added to the wells, and allowed to bind overnight at 4° C. The wells are then washed, and bound immunoglobulin is detected with HRP-conjugated anti-human IgG (Amersham Biosciences, Piscataway, N.J.) followed by development with a tetramethylbenzidine/peroxide reagent (DAKO, Carpinteria, Calif.).

### Example 10

OGFr-ARF-specific antibodies (Mollick et al., Cancer Immunity 3:3 (2003)) are conjugated to Iodine-131 using IODO-GEN (Pierce Biotechnology, Inc., Rockford, Ill.).

Human cancer patients are injected intravenously with ORIC (10 mg/kg) and the ¹³¹I-labeled OGFr-ARF antibodies once a week for three weeks. Monitoring of the tumors reveals a measurable reduction in the size of the tumors from the treated patients. In additional, the overall survival rate of treated patients increases as compared with untreated patients. Details on the administration of radiolabeled antibodies to human cancer patients can be found in Juweid, The Journal of Nuclear Medicine 43(11): 1507-1529(2002).

The complete disclosures of the patents, patent documents and publications cited herein are incorporated by reference in their entirety as if each were individually incorporated. In case of conflict, the present specification, including definitions, shall control.

Various modifications and alterations to this invention will become apparent to those skilled in the art without departing from the scope and spirit of this invention. Illustrative embodiments and examples are provided as examples only and are not intended to limit the scope of the present invention. The scope of the invention is limited only by the claims set forth as follows.

What is claimed is:

1. A method of increasing a biological activity in response to an opioid receptor ligand, the method comprising:
contacting a cell with an amount of an opioid receptor inducing compound effective for increasing expression of an opioid receptor; and
contacting the cell with an opioid receptor ligand.

2. The method of claim 1 wherein the cell is a skin cell, a blood cell, a neuron, or a cell derived from any one of the foregoing.

3. The method of claim 1 wherein the opioid receptor comprises a μ-opioid receptor or a ζ-opioid receptor.

4. The method of claim 3 wherein the opioid receptor is Opioid Growth Factor receptor.

5. The method of claim 1 wherein the opioid receptor inducing compound has a molecular weight of about 1000 Daltons or less.

6. The method of claim 5 wherein the opioid receptor inducing compound comprises an imidazoquinoline amine, a tetrahydroimidazoquinoline amine, an imidazopyridine amine, a 1,2-bridged imidazoquinoline amine, a 6,7-fused cycloalkylimidazopyridine amine, an imidazonaphthyridine amine, a tetrahydroimidazonaphthyridine amine, an oxazoloquinoline amine, a thiazoloquinoline amine, an oxazolopyridine amine, a thiazolopyridine amine, an oxazolonaphthyridine amine, a thiazolonaphthyridine amine, or a pro-drug of any of the foregoing.

7. The method of claim 1 wherein the cells are contacted with the opioid receptor inducing compound in vivo.

8. The method of claim 1 wherein the cells are contacted with the opioid receptor inducing compound in vitro.

9. The method of claim 1 wherein the ligand for the opioid receptor comprises an opioid peptide.

10. The method of claim 1 wherein the ligand for the opioid receptor is Opioid Growth Factor.

11. The method of claim 1 wherein the ligand for the opioid receptor comprises an antibody.

12. The method of claim 1 wherein the ligand for the opioid receptor comprises an antibody specific for Opioid Growth Factor receptor.

13. The method of claim 11 wherein the antibody is conjugated to a cytotoxic moiety.

14. The method of claim 1 wherein the opioid receptor is expressed in an alternate reading frame.

15. A method of treating a condition in a subject treatable by increasing expressions of an opioid receptor, the method comprising:
contacting cells capable of expressing an opioid receptor with a therapeutically effective amount of an opioid receptor inducing compound; and
contacting the cells with a therapeutically effective amount of an opioid receptor ligand.

16. The method of claim 15 wherein the cell is a skin cell, a blood cell, a neuron, or a cell derived from any one of the foregoing.

17. The method of claim 15 wherein the opioid receptor comprises a μ-opioid receptor or a ζ-opioid receptor.

18. The method of claim 17 wherein the opioid receptor is Opioid Growth Factor receptor.

19. The method of claim 15 wherein the opioid receptor inducing compound has a molecular weight of about 1000 Daltons or less.

20. The method of claim 19 wherein the opioid receptor inducing compound comprises an imidazoquinoline amine, a tetrahydroimidazoquinoline amine, an imidazopyridine amine, a 1,2-bridged imidazoquinoline amine, a 6,7-fused cycloalkylimidazopyridine amine, an imidazonaphthyridine amine, a tetrahydroimidazonaphthyridine amine, an oxazoloquinoline amine, a thiazoloquinoline amine, an oxazolopyridine amine, a thiazolopyridine amine, an oxazolonaphthyridine amine, a thiazolonaphthyridine amine, or a pro-drug of any of the foregoing.

21. The method of claim 15 wherein contacting cells capable of expressing an opioid receptor with an opioid receptor inducing compound comprises:
(a) contacting cells capable of expressing an opioid receptor with the opioid receptor inducing compound in vitro, thereby generating induced cells; and
(b) administering at least a portion of the induced cells to the subject.

22. The method of claim 21 wherein the cells capable of expressing an opioid receptor are collected from the subject.

23. The method of claim 15 wherein the cells capable of expressing an opioid receptor are contacted with the opioid receptor inducing compound in vivo.

24. The method of claim 23 wherein the cells capable of expressing an opioid receptor are contacted with the opioid receptor ligand in vivo.

25. The method of claim 23 wherein contacting cells capable of expressing an opioid receptor with an opioid receptor inducing compound comprises administering the opioid receptor inducing compound to the subject.

26. The method of claim 25 further comprising administering the opioid receptor ligand to the subject.

27. The method of claim 25 wherein administering the opioid receptor inducing compound to the subject reduces pain associated with treating the condition.

28. The method of claim 15 wherein the ligand for the opioid receptor comprises an opioid peptide.

29. The method of claim 15 wherein the ligand for the opioid receptor is Opioid Growth Factor.

30. The method of claim 15 wherein the ligand for the opioid receptor comprises an antibody.

31. The method of claim 15 wherein the ligand for the opioid receptor comprises an antibody specific for Opioid Growth Factor receptor.

32. The method of claim 30 wherein the antibody is conjugated to a cytotoxic moiety.

33. The method of claim 15 wherein the opioid receptor is expressed in an alternate reading frame.

34. A method of reducing effects of tissue damage, the method comprising:
increasing expression of an opioid receptor in cells of damaged tissue by administering an opioid receptor inducing compound to at least a portion of the damaged tissue; and
contacting the cells with an opioid receptor ligand.

35. The method of claim 34 wherein the effects of tissue damage comprise pain or scarring.

36. The method of claim 34 wherein the damaged tissue comprises skin, mucosal tissue, vascular tissue, cardiac tissue, or neuronal tissue.

37. The method of claim 34 wherein the opioid receptor inducing compound comprises an imidazoquinoline amine, a tetrahydroimidazoquinoline amine, an imidazopyridine amine, a 1,2-bridged imidazoquinoline amine, a 6,7-fused cycloalkylimidazopyridine amine, an imidazonaphthyridine amine, a tetrahydroimidazonaphthyridine amine, an oxazoloquinoline amine, a thiazoloquinoline amine, an oxazolopyridine amine, a thiazolopyridine amine, an oxazolonaphthyridine amine, a thiazolonaphthyridine amine, or a pro-drug of any of the foregoing.

38. The method of claim 34 wherein the opioid receptor ligand comprises an exogenous opioid receptor ligand.

39. A therapeutic combination comprising:
an opioid receptor inducing compound in an amount effective to induce expression of at least on opioid receptor; and
an opioid receptor ligand in an amount effective to modulate an opioid receptor-mediated biological activity.
